(19) United States
(12) Patent Application Publication
(10) Pub. No.: US 2026/0272384 A1
(43) Pub. Date: Sep. 17, 2026

(54) ARTICLE AND SENSOR SYSTEM

(71) Applicant: Solventum Intellectual Properties Company, Maplewood, MN (US)

(72) Inventors: Tony J. KAUFMAN, Rosemount, MN (US); Audrey A. SHERMAN, Woodbury, MN (US)

(73) Assignee: Solventum Intellectual Properties Company, Maplewood, MN (US)

(21) Appl. No.: 19/469,479

(22) PCT Filed: May 2, 2024
(86) PCT No.: PCT/US2024/027374

§ 371 Date: Sep. 26, 2025

Related U.S. Application Data
(60) Provisional application No. 63/500,291, filed on May 5, 2023.

(51) Int. Cl. A61B 5/00; A61B 5/01; A61B 5/024

(52) U.S. Cl. CPC A61B 5/68335; A61B 5/01; A61B 5/02438; A61B 2562/164; A61B 2562/18

(57) ABSTRACT

An article wearable on a skin of a user is provided. The article includes a base film including a first base major surface and a second base major surface opposite to the first base major surface and facing the skin of the user. The base film further includes a through-opening extending between the first base major surface and the second base major surface. The base film further includes a base adhesive layer including a base adhesive configured to removably attach the base film to the skin of the user. The through-opening is configured to align with and at least partially and removably receive a sensor therethrough upon placement of the article on the sensor. The article further includes a cover film at least partially disposed on the base film. The cover film is at least partially attached to the first base major surface of the base film.

### TECHNICAL FIELD

The present disclosure relates generally to an article wearable on a skin of a user. The present disclosure further relates to a sensor system comprising the article that is wearable on the skin of the user.

### BACKGROUND

Articles that include adhesive layers are generally found in a variety of applications. Such articles may be held against an external surface with the help of the adhesive layer. For example, medical diagnostic devices are typically held against a skin of a user through an adhesive layer. Such devices need to be attached to the external surface (e.g., the skin of the user) that is not rigid and generally not flat. Additionally, such devices tend to catch on to external obstacles and get pulled off the skin of the user, e.g., through edge lifting. Once the device falls off the user, diagnostic data may not be collected, and a replacement device may not always be available. Further, the original device may not be reattached due to sterility issues or a loss of function. Thus, articles that may last longer on the external surface (e.g., the skin of the user) may be desirable. Moreover, articles with improved wearability on the external surface are desirable.

### SUMMARY

In a first aspect, the present disclosure provides an article wearable on a skin of a user. The article includes a base film including a first base major surface and a second base major surface opposite to the first base major surface and facing the skin of the user. The base film further includes a through-opening extending between the first base major surface and the second base major surface. The base film further includes a base adhesive layer forming the second base major surface and including a base adhesive configured to removably attach the base film to the skin of the user. The through-opening is configured to align with and at least partially and removably receive a sensor therethrough upon placement of the article on the sensor that is attached to the skin of the user. The article is separate from the sensor. The article further includes a cover film at least partially disposed on the base film. The cover film includes a first cover major surface and a second cover major surface opposite to the first cover major surface and at least partially attached to the first base major surface of the base film. The base film and the cover film are configured to removably and at least partially enclose the sensor, such that the article is removable from the skin of the user while the sensor remains attached to the skin of the user.

In a second aspect, the present disclosure provides a sensor system including a sensor attached to a skin of a user. The sensor system further includes the article of the first aspect of the present disclosure. The article is separate from the sensor and removably attached to the skin of the user. The through-opening of the base film at least partially and removably receives the sensor therethrough upon placement of the article on the sensor. The article removably and at least partially encloses the sensor, such that the article is removable from the skin of the user while the sensor remains attached to the skin of the user.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a schematic perspective view of a sensor system, according to an embodiment of the present disclosure;

FIG. 2 is a schematic top view of the sensor system of FIG. 1, according to an embodiment of the present disclosure;

FIG. 3 is a schematic side view of the sensor system of FIG. 1, according to an embodiment of the present disclosure;

FIG. 4 is a schematic sectional side view of the sensor system of FIG. 1, according to an embodiment of the present disclosure;

FIG. 5 is a schematic sectional side view of a sensor system, according to another embodiment of the present disclosure;

FIG. 6 is a schematic sectional side view of a sensor system, according to another embodiment of the present disclosure;

FIG. 7 is a schematic sectional side view of a sensor system, according to another embodiment of the present disclosure; and

FIG. 8 is a schematic sectional side view of a sensor system, according to another embodiment of the present disclosure; and

FIG. 9 is a schematic top view of a sensor system, according to another embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

As used herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20 % for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A and B” should be understood to mean “only A, only B, or both A and B”.

As used herein, the recitations of numerical ranges by endpoints include all numbers subsumed within that range as well as the endpoints (e.g., 1 to 5 includes 1, 1.5, 2, 2.75, 3, 3.80, 4, 5, etc.).

As used herein, when a first material is termed as “same” or “similar” as a second material, at least 90 weight % of the first and second materials are identical and any variation between the first and second materials comprises less than about 10 weight % of each of the first and second materials.

Unless specified or limited otherwise, the terms “attached,” “connected,” “coupled”, and variations thereof, are used broadly and encompass both direct physical connections, or indirect physical connections between two or more components that are connected together by one or more additional components. For example, a first component may be coupled to a second component by being directly connected together or by being connected by a third component. In some examples, coupling, connection, and attachment may also include mechanical, thermal, electrical, or chemical coupling (such as a chemical bond) in some contexts.

As used herein, the terms “layer,” “sheet,” and “film” or variations thereof, are used to describe a component having a thickness that is small relative to its length and width.

As used herein, the term “film” generally refers to a material with a very high ratio of length or width to thickness. A film has two major surfaces defined by a length and width. Films typically have good flexibility and can be used for a wide variety of applications, including displays. Films may also be of thickness or material composition, such that they are semi-rigid or rigid. Films described in the present disclosure may be composed of various polymeric materials. Films may be monolayer, multilayer, or blend of different polymers.

The present disclosure relates to an article wearable on a skin of a user. The article includes a base film including a first base major surface and a second base major surface opposite to the first base major surface and facing the skin of the user. The base film further includes a through-opening extending between the first base major surface and the second base major surface. The base film further includes a base adhesive layer forming the second base major surface and including a base adhesive configured to removably attach the base film to the skin of the user. The through-opening is configured to align with and at least partially and removably receive a sensor therethrough upon placement of the article on the sensor that is attached to the skin of the user. The article is separate from the sensor. The article further includes a cover film at least partially disposed on the base film. The cover film includes a first cover major surface and a second cover major surface opposite to the first cover major surface and at least partially attached to the first base major surface of the base film. The base film and the cover film are configured to removably and at least partially enclose the sensor, such that the article is removable from the skin of the user while the sensor remains attached to the skin of the user.

As the cover film is at least partially disposed on the base film, the cover film may protect the base film from environmental factors, such as dirt, moisture, etc., which can otherwise adversely affect the functioning of the sensor. Moreover, the cover film may also protect the base film from catching on to external objects, such as clothing, thereby preventing the base film from being peeled up from an external surface (e.g., the skin of the user) to which the article may be attached. Longer attachment period of the article with the skin may improve a user experience and may enable less landfill waste.

Additionally, as the article is removable from the skin of the user while the sensor remains attached to the skin of the user, the article can be easily replaced with a new one when required. In this way, the sensor remains attached to the skin of the user during removal of the article from the skin of the user. In some cases, even if the article is peeled up mistakenly, there is no need to attach a new sensor to the skin of the user because the sensor remains attached to the skin of the user during removal of the article from the skin of the user. This provision may improve an overall user experience and may aid in wearability of the article on the skin of the user. Thus, the article of the present disclosure may provide a highly compliant attachment with longer attachment period and a better user experience.

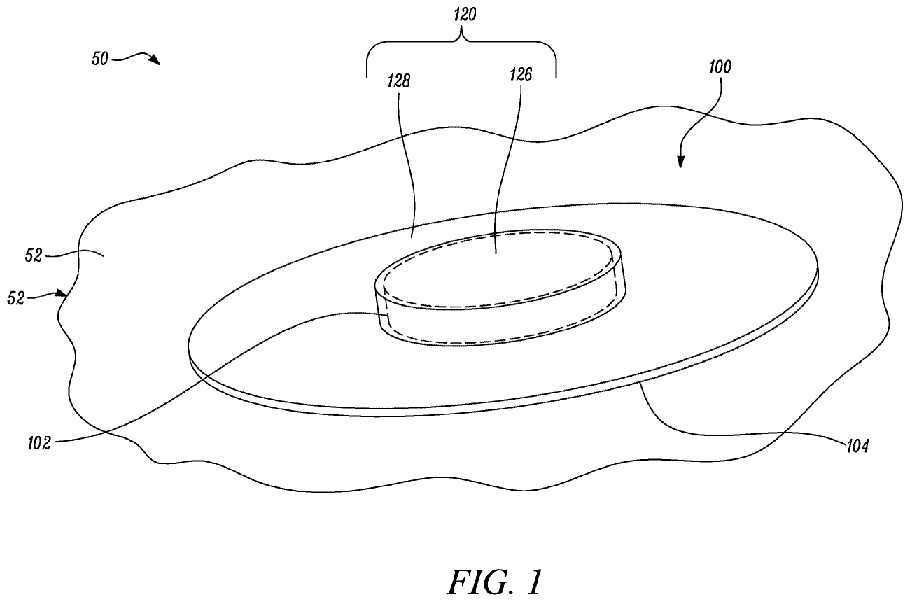

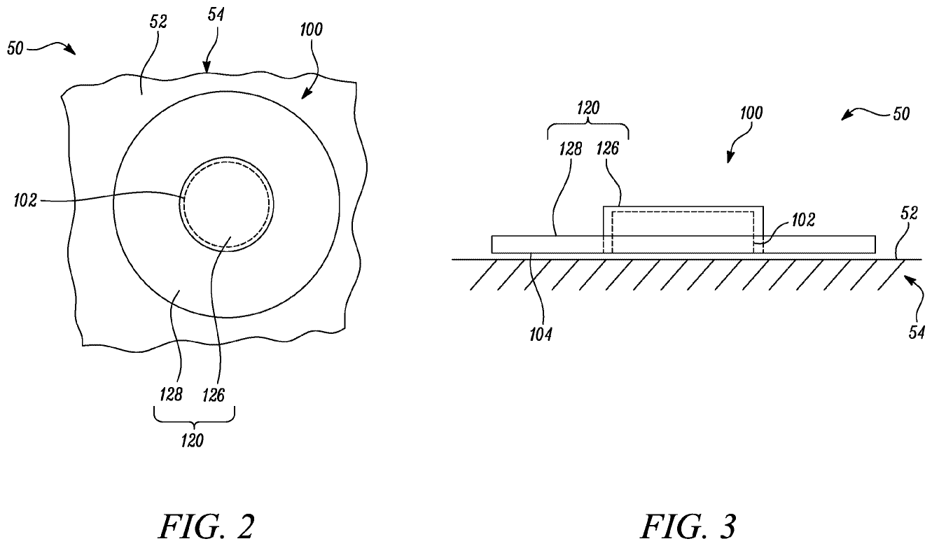

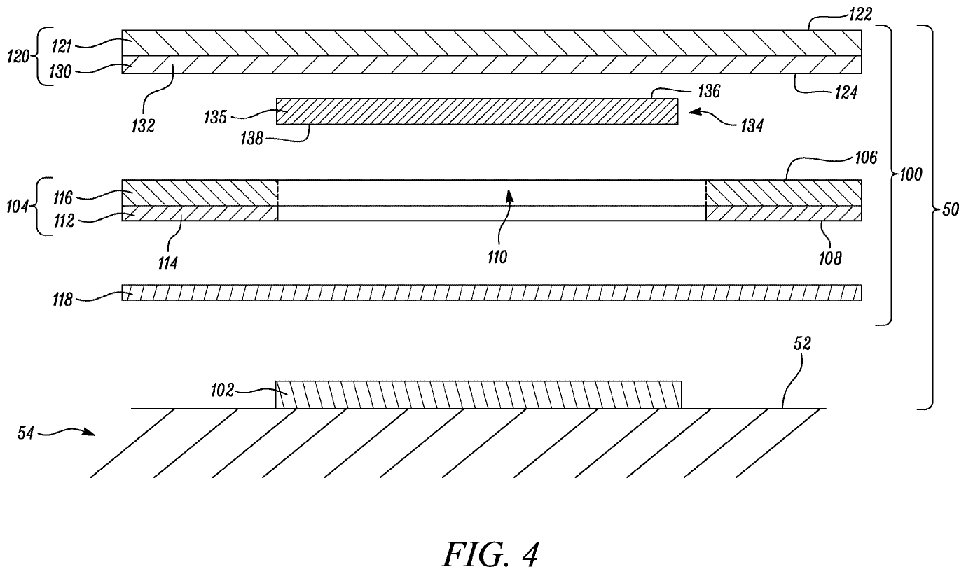

Referring now to Figures, FIG. 1 is a schematic perspective view of a sensor system 50, according to an embodiment of the present disclosure. FIG. 2 is a schematic top view of the sensor system 50, according to an embodiment of the present disclosure. FIG. 3 is a schematic side view of the sensor system 50, according to an embodiment of the present disclosure. FIG. 4 is a schematic sectional side view of the sensor system 50, according to an embodiment of the present disclosure. The sensor system 50 may include a medical diagnostic device. Referring to FIGS. 1 to 4, the sensor system 50 includes a sensor 102 attached to a skin 52 of a user 54. The sensor 102 may be adhesively attached to the skin 52 of the user 54.

In some embodiments, the sensor 102 may collect physiological data from the skin 52 of the user 54. In some embodiments, the sensor 102 may collect information relating to one or more of calories burned, steps taken, activity levels, sleep patterns, and other parameters. In some embodiments, the sensor 102 may collect one or more of the following data: skin surface temperature data of the user 54, core body temperature data of the user 54, motion or inactivity data of the user 54, body angle data of the user 54, heart rate and/or ECG data of the user 54, and respiration information of the user 54. In some embodiments, the sensor 102 may utilize software algorithms to process the collected data and determine or provide indication of one or more of sleep quality and quantity, activity level, step count, caloric expenditures, etc.

In some embodiments, the sensor 102 may include multiple sensors and/or multiple sensor types that collect data. In some embodiments, the sensor 102 may include provisions that enable wireless transfer of collected data to an external device, e.g., a computer or a mobile device. In some embodiments, the sensor 102 may, continuously or intermittently, transmit the collected data wirelessly to the external device, such that the collected data may be viewed or displayed in real time on the external device. In some embodiments, the sensor 102 may also utilize cloud-based infrastructure for storage and presentation of the collected and/or processed data.

The sensor system 50 further includes an article 100 wearable on the skin 52 of the user 54. The article 100 is separate from the sensor 102 and removably attached to the skin 52 of the user 54. As shown in FIG. 4, the article 100 includes a base film 104 including a first base major surface 106 and a second base major surface 108 opposite to the first base major surface 106 and facing the skin 52 of the user 54.

The base film 104 further includes a through-opening 110 extending between the first base major surface 106 and the second base major surface 108. The through-opening 110 is configured to align with and at least partially and removably receive the sensor 102 therethrough upon placement of the article 100 on the sensor 102 that is attached to the skin 52 of the user 54. In other words, the through-opening 110 of the base film 104 at least partially and removably receives the sensor 102 therethrough upon placement of the article 100 on the sensor 102. In some embodiments, the base film 104 is annular, such that the through-opening 110 is disposed centrally in the base film 104. In other embodiments, the base film 104 may have shapes like rectangular, square, or polygonal with the through-opening 110 disposed centrally in the base film 104.

The base film 104 further includes a base adhesive layer 112 forming the second base major surface 108 and including a base adhesive 114 configured to removably attach the base film 104 to the skin 52 of the user 54. The base adhesive layer 112 may be breathable and conformable to the skin 52 of the user 54. In some embodiments, the base adhesive 114 is an acrylic adhesive. Specifically, the base adhesive 114 may be tackified acrylic adhesive. In some embodiments, the base adhesive 114 may be a pressure sensitive adhesive or a stretch-release adhesive.

In the illustrated embodiment of FIG. 4, the base film 104 further includes a backing layer 116 attached to the base adhesive layer 112. The backing layer 116 includes a spunlace polyester nonwoven material. In other embodiments, the backing layer 116 may include a stretchable spunlace fabric. It is appreciated that various other materials can be acceptably used for the backing layer 116. For instance, the backing layer 116 may include a knitted polyester. The backing layer 116 forms the first base major surface 106 of the base film 104. The through-opening 110 extends through the backing layer 116 and the base adhesive layer 112.

In some embodiments, the article 100 further includes a release liner 118 releasably attached to and covering the base adhesive 114 of the base film 104. The release liner 118 is removed from the base adhesive 114 before removably attaching the base adhesive 114 to the skin 52 of the user 54. The release liner 118 may be a film formed from paper; rubber; various foils such as aluminum foil, copper foil, stainless steel foil, iron foil, duralumin foil, tin foil, titanium foil, gold foil; film made of various resins such as polyethylene, polypropylene, polyvinyl chloride, polyester, polyamide; foams such polyurethane foam, vinyl foam, polyethylene foam, styrene foam; nonwoven fabric; woven fabric; felt, or a laminate of these materials with a polymer material may be used as a base material.

In some embodiments, the release liner 118 may include a microstructured surface. The release liner 118 remains attached with the base film 104 during shipment and processing and is only removed when the article 100 is to be used. In this way, the base film 104 is protected until the article 100 is to be used. A wide range of microstructured release liners are suitable. Typically, the microstructured release liner 118 is prepared by embossing. This means that the release liner 118 has an embossable surface which is contacted to a structured tool with the application of pressure and/or heat to form an embossed surface.

The article 100 further includes a cover film 120 at least partially disposed on the base film 104. The cover film 120 includes a first cover major surface 122 and a second cover major surface 124 opposite to the first cover major surface 122. The second cover major surface 124 is at least partially attached to the first base major surface 106 of the base film 104. In the illustrated embodiment of FIG. 4, the first cover major surface 122 of the cover film 120 at least partially forms an exterior surface of the article 100. The cover film 120 includes a substrate cover layer 121 forming the first cover major surface 122 of the cover film 120.

Further, the cover film 120 is a printable aesthetic film. Specifically, the cover film 120 is the printable aesthetic film having a matte finish. Specifically, the substrate cover layer 121 is a printable aesthetic film. The first cover major surface 122 may include at least one of a logo, a serial number, a text, a graphical design, and a graphical image. In some embodiments, the cover film 120 includes polyurethane. In other embodiments, the cover film 120 may include polyurethane foam to provide cushioning, moldability, and support to the article 100. In some embodiments, the cover film 120 further includes a raised central portion 126 aligned with the through-opening 110 and a peripheral portion 128 surrounding the raised central portion 126 and attached to the base film 104. The raised central portion 126 and the peripheral portion 128 together enclose and surround the sensor 102. In some embodiments, the raised central portion 126 may include perforations (not shown). The perforations may extend from the first cover major surface 122 to the second cover major surface 124 so as to create a pathway allowing egress of any moisture or sweat near the sensor 102. In some embodiments, the base adhesive layer 114 may include a microstructured surface (not shown) with at least one channel extending to at least one boundary of the base adhesive layer 114. Such microstructured surface is also provided in US Patent Publication No. 2022/0257424. The at least one channel may permit transport of fluid (such as moisture or sweat) from the sensor 102 to the first cover major surface 122. Alternatively, in some embodiments, the cover film 120 may be devoid of through-openings. Moreover, when the release liner 118 is removed prior to the application of the article 100 to the skin 52 of the user 54, the corresponding opposite microstructural features are imparted to the base adhesive layer 114.

In the illustrated embodiment of FIG. 4, the cover film 120 further includes a lower adhesive layer 130 forming the second cover major surface 124. The lower adhesive layer 130 is attached to the substrate cover layer 121. The lower adhesive layer 130 includes a lower adhesive 132 that adhesively bonds the cover film 120 to the base film 104. Specifically, in the embodiment shown in FIG. 4, the lower adhesive 132 adhesively bonds the second cover major surface 124 of the cover film 120 to the first base major surface 106 of the base film 104. The base film 104 and the cover film 120 are configured to removably and at least partially enclose the sensor 102, such that the article 100 is removable from the skin 52 of the user 54 while the sensor 102 remains attached to the skin 52 of the user 54. In other words, the 100 article removably and at least partially encloses the sensor 102, such that the article 100 is removable from the skin 52 of the user 54 while the sensor 102 remains attached to the skin 52 of the user 54.

As the cover film 120 is at least partially disposed on the base film 104, the cover film 120 may protect the base film 104 from environmental factors, such as dirt, moisture, etc., which can otherwise adversely affect functioning of the sensor 102. Moreover, the cover film 120 may also protect the base film 104 from catching on to external objects, such as clothing, thereby preventing the base film 104 from being peeled up from an external surface (e.g., the skin 52 of the user 54) to which the article 100 may be attached. Longer attachment period of the article 100 with the skin 52 may improve a user experience and may enable less landfill waste.

Additionally, as the article 100 is removable from the skin 52 of the user 54 while the sensor 102 remains attached to the skin 52 of the user 54, the article 100 can be easily replaced with a new one when required. In this way, the sensor 102 remains attached to the skin 52 of the user 54 during removal of the article 100 from the skin 52 of the user 54. In some cases, even if the article 100 is peeled up mistakenly, there is no need to attach a new sensor to the skin 52 of the user 54 because the sensor 102 remains attached to the skin 52 of the user 54 during removal of the article 100 from the skin 52 of the user 54. This provision may improve an overall user experience and may aid in wearability of the article 100 on the skin 52 of the user 54. Thus, the article 100 may provide a highly compliant attachment with longer attachment period and a better user experience.

With continued reference to FIG. 4, the article 100 further includes an intermediate film 134 disposed between the cover film 120 and the base film 104. The intermediate film 134 includes a first intermediate major surface 136 adhesively bonded to the second cover major surface 124 of the cover film 120. Specifically, the lower adhesive 132 adhesively bonds the cover film 120 to the intermediate film 134. Thus, the lower adhesive 132 adhesively bonds the cover film 120 to each of the intermediate film 134 and the base film 104. The intermediate film 134 further includes a second intermediate major surface 138 opposite to the first intermediate major surface 136 and aligned with the through-opening 110 of the base film 104. The second intermediate major surface 138 is adhesive-free and is configured to removably engage the sensor 102, such that the intermediate film 134 is removable from the sensor 102 while the sensor 102 remains attached to the skin 52 of the user 54. Due to adhesive-free second intermediate major surface 138, none of the films of the article 100 remain attached to the sensor 102 upon removal of the article 100 from the skin 52 of the user 54. Therefore, when required, the article 100 can be easily replaced with a new one without displacing the sensor 102 attached to the skin 52 of the user 54. In the illustrated embodiment of FIG. 4, the intermediate film 134 includes a substrate intermediate layer 135 forming the first intermediate major surface 136 as well as the second intermediate major surface 138 opposite to the first intermediate major surface 136. In some embodiments, the intermediate film 134 may include perforations (not shown) extending from the first intermediate major surface 136 to the second intermediate major surface 138.

In some embodiments, shape and dimensions of the second intermediate major surface 138 of the intermediate film 134 are substantially the same as cross-sectional shape and dimensions of the sensor 102. Specifically, length and breadth of the intermediate film 134 are substantially the same as respective length and breadth of the sensor 102. Further, in some embodiments, the shape and dimensions of the second intermediate major surface 138 of the intermediate film 134 are substantially the same as the cross-sectional shape and dimensions of the through-opening 110. The second intermediate major surface 138 of the intermediate film 134 can therefore be easily aligned with the sensor 102 and the through-opening 110. In some embodiments, the intermediate film 134 includes polyethylene. In some embodiments, the intermediate film 134 is a low-density polyethylene film. The intermediate film 134 may have a thickness between 0.1 millimeters (mm) to 0.15 mm. The intermediate film 134 may have a translucent matte finishing. The intermediate film 134 may be made of a water resistant and heat sealable material.

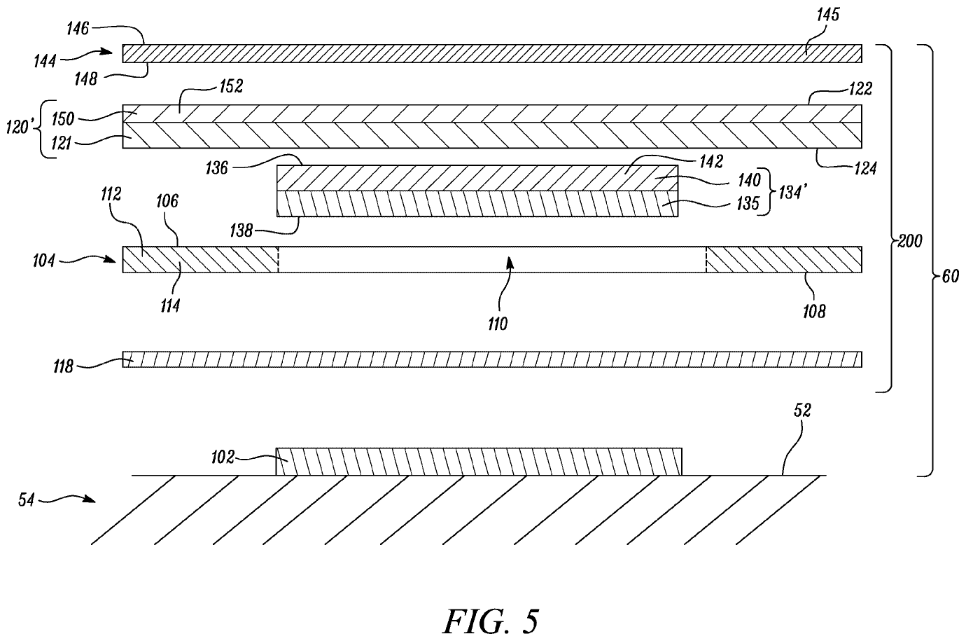

FIG. 5 is a schematic sectional side view of a sensor system 60, according to another embodiment of the present disclosure. The sensor system 60 is substantially similar to the sensor system 50 of FIG. 4, with common components being referred to by same numerals. However, the sensor system 60 includes an article 200 (instead of the article 100 shown in FIG. 4). The article 200 is substantially similar to the article 100 of FIG. 4, with common components being referred to by same numerals. A functional advantage of the article 200 is substantially the same as that of the article 100 of FIG. 4.

In the article 200, the base film 104 does not include a backing layer (i.e., the backing layer 116 shown in FIG. 4). In the article 200, the base film 104 is a dual-sided adhesive film, such that the base adhesive layer 112 further forms the first base major surface 106 of the base film 104. Therefore, in the article 200, the base adhesive layer 112 forms each of the first base major surface 106 and the second base major surface 108.

Further, the article 200 includes an intermediate film 134′ which is substantially similar to the intermediate film 134 of the article 100 shown in FIG. 4. However, the intermediate film 134′ further includes an intermediate adhesive layer 140 forming the first intermediate major surface 136. The intermediate adhesive layer 140 is attached to the substrate intermediate layer 135. The substrate intermediate layer 135 forms the second intermediate major surface 138. In some embodiments, the intermediate adhesive layer 140 may include a microstructured surface (not shown) with at least one channel extending to at least one boundary of the intermediate adhesive layer 140.

Further, the article 200 includes a cover film 120′ which is substantially similar to the cover film 120 shown in the article of FIG. 4. However, the cover film 120′ does not include a lower adhesive layer (i.e., the lower adhesive layer 130 shown in FIG. 4). In the article 200, the cover film 120′ includes an upper adhesive layer 150 forming the first cover major surface 122. The intermediate adhesive layer 140 includes an intermediate adhesive 142 that adhesively bonds the intermediate film 134′ to the second cover major surface 124 of the cover film 120′. Thus, the intermediate adhesive 142 adhesively bonds the intermediate film 134′ to the cover film 120′.

The article 200 further includes a top film 144. The top film 144 includes a first top major surface 146 at least partially forming an exterior surface of the article 200. The top film 144 further includes a second top major surface 148 opposite to the first top major surface 146 and attached to the first cover major surface 122 of the cover film 120′. Therefore, the first cover major surface 122 of the cover film 120′ does not form the exterior surface of the article 200. In some embodiments, the top film 144 is a printable aesthetic film. Specifically, the top film 144 is the printable aesthetic film having a matte finish. The first top major surface 146 may include at least one of a logo, a serial number, a text, a graphical design, and a graphical image. In some embodiments, the top film 144 includes polyurethane. In other embodiments, the top film 144 may include polyurethane foam to provide cushioning, moldability, and support to the article 200. In the illustrated embodiment of FIG. 5, the top film 144 includes a substrate top layer 145 forming the first top major surface 146 as well as the second top major surface 148 opposite to the first top major surface 146. In some embodiments, the substrate top layer 145 includes polyurethane.

The upper adhesive layer 150 includes an upper adhesive 152 that adhesively bonds the cover film 120′ to the second top major surface 148 of the top film 144. Thus, the second top major surface 148 is adhesively attached to the first cover major surface 122 of the cover film 120′ by the upper adhesive 152 of the upper adhesive layer 150. Further, in the article 200, the second cover major surface 124 is adhesive-free. In some embodiments, the cover film 120′ includes closed-cell foam. Specifically, the substrate cover layer 121 of the cover film 120′ includes closed-cell foam. In the article 200, the cover film 120′ may have a thickness between 0.4 millimeters (mm) to 0.8 mm.

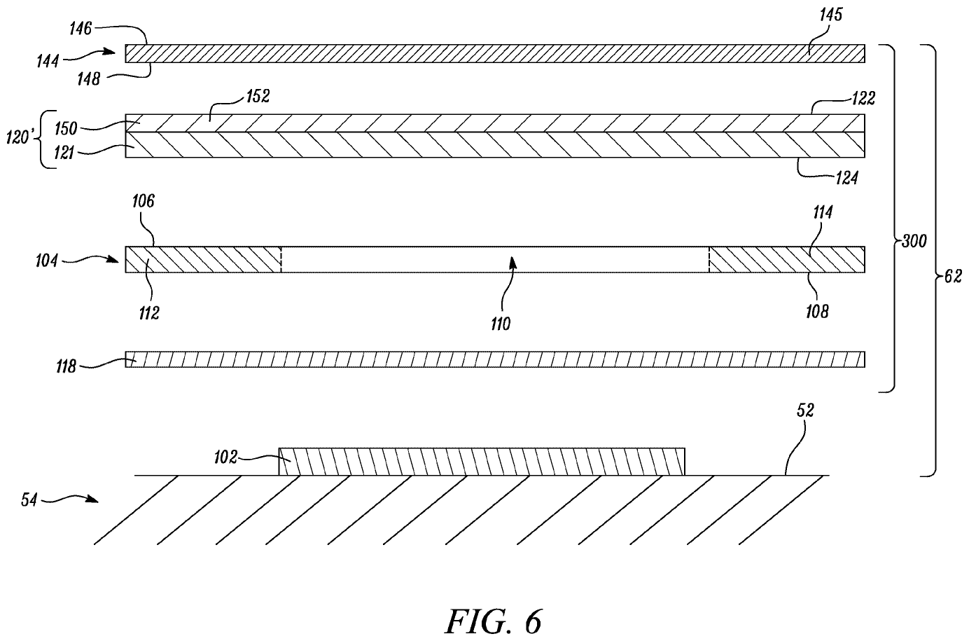

FIG. 6 is a schematic sectional side view of a sensor system 62, according to another embodiment of the present disclosure. The sensor system 62 is substantially similar to the sensor system 60 of FIG. 5, with common components being referred to by same numerals. However, the sensor system 62 includes an article 300 (instead of the article 200 shown in FIG. 5). The article 300 is substantially similar to the article 200 of FIG. 5, with common components being referred to by same numerals. A functional advantage of the article 300 is substantially the same as that of the article 200 of FIG. 5.

In the article 300, there is no intermediate film (i.e., the intermediate film 134′ shown in FIG. 5). Further, in the article 300, the second cover major surface 124 is adhesive-free and is configured to removably engage the sensor 102, such that the cover film 120′ is removable from the sensor 102 while the sensor 102 remains attached to the skin 52 of the user 54.

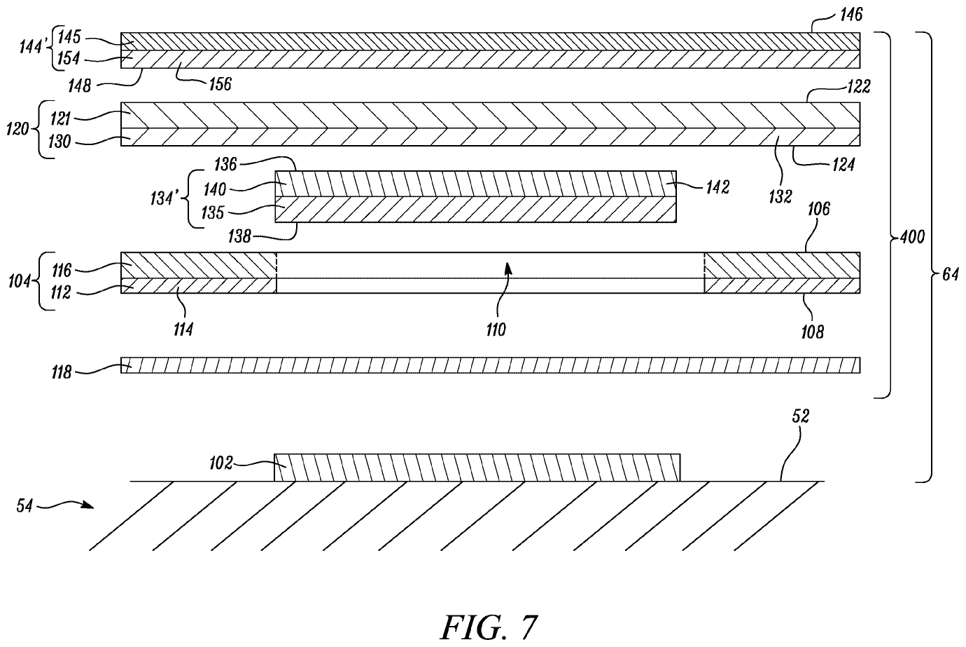

FIG. 7 is a schematic sectional side view of a sensor system 64, according to another embodiment of the present disclosure. The sensor system 64 is substantially similar to the sensor system 60 of FIG. 5, with common components being referred to by same numerals. However, the sensor system 64 includes an article 400 (instead of the article 200 shown in FIG. 5). The article 400 is substantially similar to the article 200 of FIG. 5, with common components being referred to by same numerals. A functional advantage of the article 400 is substantially the same as that of the article 200 of FIG. 5.

In the article 400, the base film 104 is not a dual-sided adhesive film like the one shown in FIG. 5. Therefore, in the article 400, the base adhesive layer 112 does not form the first base major surface 106 of the base film 104. The base film 104 in the article 400 is similar to the one which is illustrated in the article 100 of FIG. 4. Thus, in the article 400, the base film 104 includes the backing layer 116 attached to the base adhesive layer 112 and the backing layer 116 forms the first base major surface 106 of the base film 104. Further, the article 400 includes the cover film 120 (instead of the cover film 120′ shown in FIG. 5) that is also shown in FIG. 4.

The article 400 includes a top film 144′ which is substantially similar to the top film 144 shown in FIG. 5. However, the top film 144′ further includes a top adhesive layer 154 forming the second top major surface 148. The top adhesive layer 154 is attached to the substrate top layer 145. The substrate top layer 145 forms the first top major surface 146 of the top film 144′. The top adhesive layer 154 includes a top adhesive 156 that adhesively bonds the top film 144′ to the first cover major surface 122 of the cover film 120.

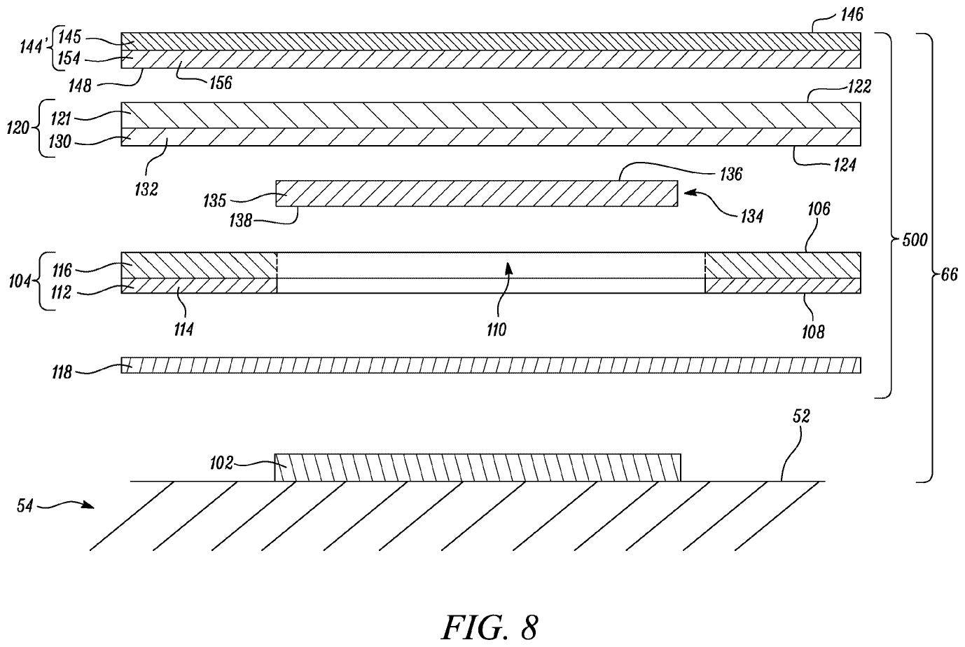

FIG. 8 is a schematic sectional side view of a sensor system 66, according to another embodiment of the present disclosure. The sensor system 66 is substantially similar to the sensor system 64 of FIG. 7, with common components being referred to by same numerals. However, the sensor system 66 includes an article 500 (instead of the article 400 shown in FIG. 7). The article 500 is substantially similar to the article 400 of FIG. 7, with common components being referred to by same numerals. A functional advantage of the article 500 is substantially the same as that of the article 400 of FIG. 7. However, the article 500 includes the intermediate film 134 (instead of the intermediate film 134′ shown in FIG. 7) which is also shown in the article 100 of FIG. 4.

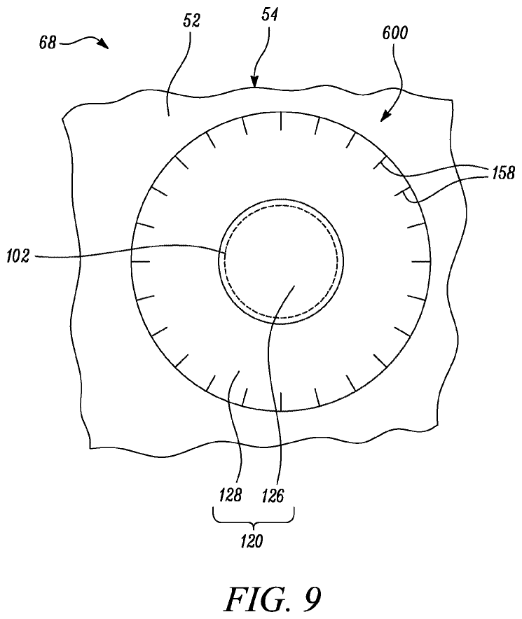

FIG. 9 is a schematic top view of a sensor system 68, according to an embodiment of the present disclosure. The sensor system 68 is substantially similar to the sensor system 50 of FIG. 2, with common components being referred to by same numerals. However, the sensor system 68 includes an article 600 (instead of the article 100 shown in FIG. 2). The article 600 is substantially similar to the article 100 of FIG. 2, with common components being referred to by same numerals. However, in the article 600, the cover film 120 includes a plurality of slits 158 disposed near the periphery of the peripheral portion 128. In some examples, the plurality of slits 158 may be through cuts. The plurality of slits 158 may enhance fluid and moisture transport in the article 600. Moreover, the plurality of slits 158 may improve conformability when the article 600 is worn by the user 54. Such plurality of slits 158 is also provided in U.S. Pat. No. 10,940,049.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. An article wearable on a skin of a user, the article comprising:
a base film, comprising:
a first base major surface;
a second base major surface opposite to the first base major surface and facing the skin of the user;
a through-opening extending between the first base major surface and the second base major surface; and
a base adhesive layer forming the second base major surface and comprising a base adhesive configured to removably attach the base film to the skin of the user,
wherein the through-opening is configured to align with and at least partially and removably receive a sensor therethrough upon placement of the article on the sensor that is attached to the skin of the user, wherein the article is separate from the sensor; and
a cover film at least partially disposed on the base film, the cover film comprising:
a first cover major surface; and
a second cover major surface opposite to the first cover major surface and at least partially attached to the first base major surface of the base film;
wherein the base film and the cover film are configured to removably and at least partially enclose the sensor, such that the article is removable from the skin of the user while the sensor remains attached to the skin of the user.

2. The article of claim 1, further comprising a top film, comprising:
a first top major surface at least partially forming an exterior surface of the article; and
a second top major surface opposite to the first top major surface and attached to the first cover major surface of the cover film.

3. The article of claim 2, wherein the cover film comprises an upper adhesive layer forming the first cover major surface and comprising an upper adhesive that adhesively bonds the cover film to the second top major surface of the top film, and wherein the second cover major surface is adhesive-free and is configured to removably engage the sensor, such that the cover film is removable from the sensor while the sensor remains attached to the skin of the user.

4. The article of claim 3, wherein the base film is a dual-sided adhesive film, such that the base adhesive layer further forms the first base major surface of the base film.

5. The article of claim 2, wherein the top film is a printable aesthetic film.

6. The article of claim 2, wherein the top film comprises polyurethane.

7. The article of claim 2, wherein the top film comprises a top adhesive layer forming the second top major surface and comprising a top adhesive that adhesively bonds the top film to the first cover major surface of the cover film.

8. The article of claim 1, further comprising an intermediate film disposed between the cover film and the base film, wherein the intermediate film comprises a first intermediate major surface adhesively bonded to the second cover major surface of the cover film and a second intermediate major surface opposite to the first intermediate major surface and aligned with the through-opening of the base film, wherein the second intermediate major surface is adhesive-free and is configured to removably engage the sensor, such that the intermediate film is removable from the sensor while the sensor remains attached to the skin of the user.

9. The article of claim 8, wherein the cover film comprises a lower adhesive layer forming the second cover major surface and comprising a lower adhesive that adhesively bonds the cover film to each of the intermediate film and the base film.

10. The article of claim 8, wherein the intermediate film comprises an intermediate adhesive layer forming the first intermediate major surface and comprising an intermediate adhesive that adhesively bonds the intermediate film to the second cover major surface of the cover film.

11. The article of claim 8, wherein the intermediate film comprises polyethylene.

12. The article of claim 1, wherein the first cover major surface of the cover film at least partially forms an exterior surface of the article.

13. The article of claim 12, wherein the cover film is a printable aesthetic film.

14. The article of claim 12, wherein the cover film comprises polyurethane.

15. The article of claim 1, wherein the base adhesive is an acrylic adhesive.

16.-21. (canceled)
